(19) United States
(12) Patent Application Publication
(10) Pub. No.: US 2003/0171690 A1
(43) Pub. Date: Sep. 11, 2003

(54) Enhanced electronic nasolacrimal intubation

(76) Inventors: Peter Michalos, Southampton, NY (US); Arie Michalos, Southampton, NY (US)

Correspondence Address: NOTARO AND MICHALOS, 100 DUTCH HILL ROAD, SUITE 110, ORANGEBURG, NY, 10962-2100, US

(21) Appl. No.: 10/090,705

(22) Filed: Mar. 5, 2002

(51) Int. Cl. A61B 5/05

(52) U.S. Cl. 600/547

(57) ABSTRACT

An apparatus facilitates location of an electrically conductive probe extending through a body passage, using a tool. The apparatus includes a circuit which is normally open and which includes a signaling mechanism and power source for powering the signaling mechanism to generate a signal that can be perceived by a practitioner when the circuit is closed. Clips or other connectors attach the circuit to conductive portions of the probe and the tool. The circuit is closed when the tool touches the probe to generate the signal and inform the practitioner that the probe end has been found.

### CROSS-REFERENCE TO DISCLOSURE DOCUMENT

Reference is made to Disclosure Document No. 489936 received at the U.S. PTO on Mar. 5, 2001 and disclosing the present invention.

### FIELD AND BACKGROUND OF THE INVENTION

The present invention relates generally to the field of medicine and, in particular, to a new and useful method and apparatus for enhanced intubation of the lacrimal ducts and the like.

Lacrimal fluids or “tears” are normally supplied continuously to the eye socket from the lacrimal gland. Normally, excess fluid is drained through canaliculi or small passageways commencing adjacent the inner corner of the eye. The fluid is collected in the lacrimal sac and then drained via the nasolacrimal duct which leads the fluid to the inferior nasal meatus.

If the lacrimal ducts become blocked, the fluid can no longer flow to the nasal meatus. Such closure can result from congenital anomalies, accidents, inflammation and advanced aging, as well as other physiological conditions. The result is that the eye is continually brimming over with fluid causing discomfort to the patient.

One approach to correcting a blocked lacrimal duct is described in U.S. Pat. No. 4,380,239 to Crawford, et al. issued Apr. 19, 1983 for Intubation of Lacrimal Ducts. This patent to Crawford, et al. is incorporated here by reference for its teaching of an apparatus and method of intubating a lacrimal duct. Also see the article J. S. Crawford, “Intubation of Obstrustions in the Lacrimal System,” in Can. J. Ophthalmology, 1977;12:289-292.

U.S. Pat. Nos. 4,305,395 and 5,437,625 disclose related apparatuses and methods.

Nasolacrimal intubation has been a standard of care for severe epiphora, failed pediatric probings and irrigations as well as an adjunct to dacriocystorhinostomy. The challenges of retrieving the metal portion of the tubes as they descend in the posterior nasal cavity have resulted in various methods and devices. Because of the posterior exit of the nasolacrimal system adjacent very willing to bleed mucosa and turbinates, visibility and thus tube retrieval may be difficult. Many areas of the world still use a clamp to probe blindly in the nose to the other end of the tube. The advent of nasal endoscopy has assisted those familiar with the instruments and techniques tremendously.

A need remains for improvements in this field.

### SUMMARY OF THE INVENTION

It is an object of the present invention to provide an apparatus or method for facilitating capture of the end of a probe extending through a body passage, using a tool, the apparatus and method including means for attaching a circuit to conductive portions of the probe and tool, the circuit being normally open and containing a signaling mechanism and power source for powering the signaling mechanism to generate an audible, visual and/or other signal that can be perceived by a practitioner when the circuit is closed, the circuit being closed when the tool touches the probe to generate the signal.

Accordingly, another object of the present invention is to allow the practitioner who uses such an apparatus or method to quickly determine when the tool has been used to successfully locate the probe, which can then be used to pull the probe from the passage.

One clip goes on the probe which is in the form of a metal tube entering the nasolacrimal system.

Another clip is attached on the tool or retrieval hook. Once contact is made between the metal retrieval hook and the metal tube, the circuit is completed (closed) and a low voltage and harmless buzzer or other signal in the circuit confirms the location and allows one to feel and hear where he or she is. The inventor was able to locate the tube in 45 seconds on the first try, in one case of a physician volunteer.

This simple apparatus and method helps in areas of the world where nasal endoscope is not readily available by equipment or training. This simple device and method adds yet another option to experienced as well as neophyte nasolacrimal surgeons. The combination of auditory feedback from the completed circuit enhanced proprioceptive sense of anatomy, facilitating nasolacrimal intubation.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which a preferred embodiment of the invention is illustrated.

### BRIEF DESCRIPTION OF THE DRAWINGS

In the drawings:

FIG. 1 a perspective view of a probe set and tool used on a patient, with the improvement of the present invention attached; and

FIG. 2 is a view similar to FIG. 1 of the probe being withdrawn by the tool.

### DESCRIPTION OF THE PREFERRED EMBODIMENTS

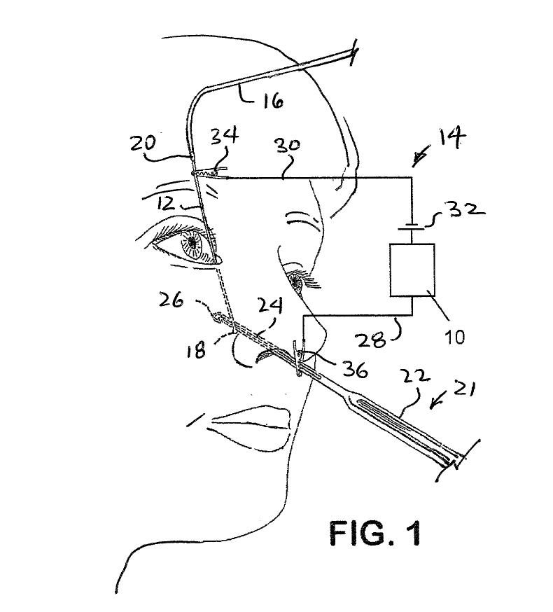

Referring now to the drawings, FIG. 1 illustrates parts of a canaliculus intubation set and tool such as that disclosed in the Crawford et al. patent cited above.

Although the complete set comprises a pair of thin metal probes, only probe 12 is shown. In a complete set, probe 12 and a like second probe are fitted to respective ends of a tube 16 of silicone rubber which is limp and flexible.

Probe 12 includes an enlarged distal end portion or “olive” 18 and a tapered enlargement 20 adjacent its proximal end. End portion 18 is rounded and the enlargement 20 has an external size comparable to that of the tube 16. The preferred probes are formed from a relatively fine tempered steel wire which is electrically conductive.

The probe can move through the patient's lacrimal duct when pushed downwardly. The end portion 18 prevents the fine wire from puncturing the patients tissue and is made, for example, from solder attached to the end of the probe and the enlargement 20 is formed in similar fashion. The end portion 18 is generally spherical so that there is a limited possibility of damage caused by the end of the probe being forced to penetrate or scratch soft tissue.

The probe is used with a tool 21 having a handle portion 22 with an aligned extension 24 terminating in a hook portion 26. The extension and hook portions 24,26 are formed of stiff, electrically conductive wire and the hook includes a recess which extends forwardly and inwardly. The recess has a width corresponding generally to the diameter of the probe 12.

In use, the patient has received probe 12 which has been pushed downwardly through the lacrimal duct and into the inferior meatus. At this point, the end portion 18 of the probe is in engagement with the lower wall of the meatus and the probe must be deflected to allow the end portion to be withdrawn.

The probe can be deflected without undue force resulting in damage to surrounding tissue, using the tool 21 to withdraw the end of the probe 12 from the nasal passage, but the practitioner must first locate and “hook” the probe.

The present invention expedites this task.

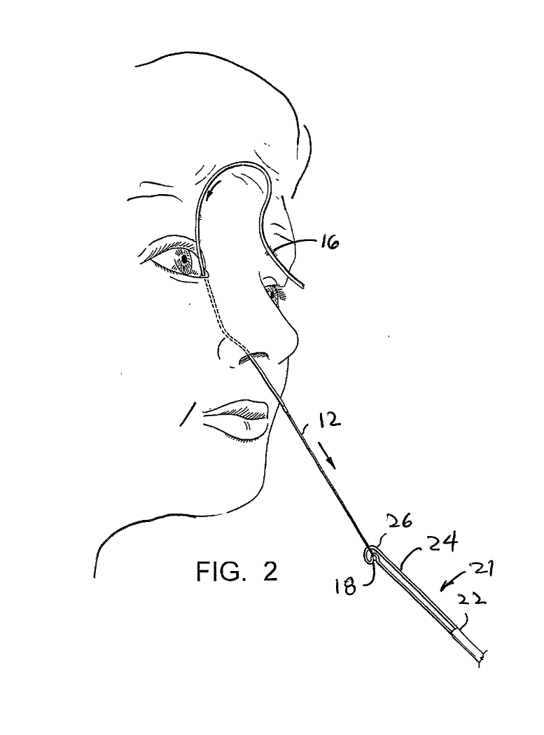

As indicated in FIG. 1, the tool 21 enters the nasal passage with the extension 24 projecting inwardly. Since it is difficult or impossible to see into the nasal passage, the usual practice is to feel around for the probe 12, and, once found to gently withdraw the tool to hook the end portion or “olive” 18 in the recess of hook portion 26. The tool is then withdrawn with the end portion 18 trapped in the recess so that the tool can be used to draw the probe through the nasal passage and outwardly as illustrated in FIG. 2.

This draws the tube 16 into the lacrimal duct. The process can be repeated using the other probe (not shown) in the opposite canaliculus outflow system and after intubation of both systems the tube is cut leaving a portion in the canaliculus systems. The tube is then left in place for a period determined by the type of damage being corrected or repaired and then subsequently removed.

According to the present invention, a circuit 14 shown in FIG. 1, is provide. The circuit included a pair of wires 28 and 30, connected to opposite contacts of a signal generating mechanism 10. Each wire has a free end connected to an alligator clip or other means 34 and 36 for attaching the wire to a conductive part of the probe 12 and tool 21. A battery or other power source or supply 32 sufficient to power the signal generating mechanism 10, is provided at any appropriate location in the circuit 14.

Mechanism 10 may, for example, be a low voltage buzzer, tone generator, LED (light-emitting-diode), or other device that can generate an audible, visual or other signal that can be perceived by a practitioner, when the circuit 14 is closed to connect the power supply or source 32 to the signal mechanism 10. Mechanism 10 may even generate both audible and visual signals, e.g. a tone and a rapidly blinking light, to advise the practitioner that he or she has made contact between the tool and the probe, even when the practitioner is not directly looking at the signaling mechanism, or when there is other noise in the operating location.

Power source 32 may be a battery holder for receiving a battery, within the meaning of this disclosure since the battery will normally be a consumable and replaceable part used with the apparatus of the invention.

The compact size and simple, and cheap construction of circuit 14 makes it readily available to practitioners in any country.

Although attachable means in the form of clips 34 and 36 are illustrated, the circuit of the present invention can be incorporated into the tool 21, which may contain the signal generating mechanism 10 and battery 32 along with a single wire or lead 30 and clip 34 for engaging a conductive part of probe 12, which is still outside the patient.

In addition, attachment means other than clips and of any known type can be utilized for connecting the circuit 14 to conductive portions of the tool and probe.

Contact between the tool extension 24 and the probe 12 amounts to the closing of a simple switch for circuit 14, but in this simple action, gives the practitioner a quick and easy way of finding the end of probe 12.

The present invention can also be adapted to other medical procedures utilizing probes extending through body passages where the need exists for locating one end of the probe.

While a specific embodiment of the invention has been shown and described in detail to illustrate the application of the principles of the invention, it will be understood that the invention may be embodied otherwise without departing from such principles.

What is claimed is:

1. An apparatus for facilitating location of an electrically conductive probe extending through a body passage, using a tool, the apparatus comprising:
a circuit which is normally open and which includes a signaling mechanism and power source for powering the signaling mechanism to generate a signal that can be perceived by a practitioner when the circuit is closed; and
means for attaching the circuit to conductive portions of the probe and the tool, the circuit being closed when the tool touches the probe to generate the signal.

2. An apparatus according to claim 1, wherein the signaling mechanism generates at least one of an audible and a visual signal.

3. An apparatus according to claim 2, wherein the means for attaching comprises a pair of clips.

4. An apparatus according to claim 3, wherein the circuit comprises a pair of wires connected to the signaling mechanism, one of the pair of clips connected to each wire, and the power source being in one of the wires.

5. An apparatus according to claim 4, wherein the power source comprises a battery holder.

6. In a probe set and tool for use in canaliculus intubation of a lacrimal duct, the probe set having a probe for passing from a nasolacrimal duct to a nasal inferior meatus, the probe having an enlarged end portion, and a tool to draw the probe from the lacrimal duct, the improvement comprising:
a circuit which is normally open and which includes a signaling mechanism and power source for powering the signaling mechanism to generate a signal that can be perceived by a practitioner when the circuit is closed; and
means for attaching the circuit to conductive portions of the probe and the tool, the circuit being closed when the tool touches the probe to generate the signal.

7. The improvement of claim 6, wherein the signaling mechanism generates at least one of an audible and a visual signal.

8. The improvement of claim 7, wherein the means for attaching comprises a pair of clips.

9. The improvement of claim 8, wherein the circuit comprises a pair of wires connected to the signaling mechanism, one of the pair of clips connected to each wire, and the power source being in one of the wires.

10. The improvement of claim 9, wherein the power source comprises a battery holder.

11. A method for facilitating location of an electrically conductive probe extending through a body passage, using a tool, the method comprising the steps of:
providing a circuit which is normally open and which includes a signaling mechanism and power source for powering the signaling mechanism to generate a signal that can be perceived by a practitioner when the circuit is closed; and
attaching the circuit to conductive portions of the probe and the tool, the circuit being closed when the tool touches the probe to generate the signal.
